(19) United States
(12) Patent Application Publication
(10) Pub. No.: US 2021/0290242 A1
(43) Pub. Date: Sep. 23, 2021

(54) Programmable Medical Wire System and Method with Emitter and Detector

(71) Applicants: Baylor University, Waco, TX (US); Scott & White Healthcare, Temple, TX (US); Board of Supervisors of Louisiana State University and Agricultural and Mechanical College, Baton Rouge, LA (US)

(72) Inventors: Keith E. Schubert, China Spring, TX (US); Linda Olafsen, Waco, TX (US); Jeffrey Olafsen, Waco, TX (US); Sunghwan Lee, West Lafayette, IN (US); Jason H. Huang, Temple, TX (US); Samantha Dayawansa, Temple, TX (US); Jin-Woo Choi, Baton Rouge, LA (US)

(73) Assignees: Baylor University, Waco, TX (US); Scott & White Healthcare, Temple, TX (US); Board of Supervisors of Louisiana State University and Agricultural and Mechanical College, Baton Rouge, LA (US)

(21) Appl. No.: 17/343,185

(22) Filed: Jun. 9, 2021

Related U.S. Application Data
(63) Continuation-in-part of application No. 16/269,689, filed on Feb. 7, 2019.
(60) Provisional application No. 62/628,614, filed on Feb. 9, 2018.

(51) Int. Cl. A61B 17/12; A61B 17/32

(52) U.S. Cl. CPC A61B 17/1214; A61B 2017/00415; A61B 17/320016; A61B 17/12113

(57) ABSTRACT

A programmable medical wire system includes a programmable wire assembly that includes: a core conductor; at least one actuator conductor electrically coupled to the core conductor, the actuator conductor being programmed to move toward a predetermined shape based on actuation; at least one selective conductor electrically coupled to the actuator conductor and configured to be electrically energized to actuate the actuator conductor to move toward the predetermined shape; at least one emitter configured to produce electromagnetic radiation of a given wavelength coupled to the programmable wire assembly; and at least one detector configured to receive emissions of the emitter and communicate signals of the emission remotely from the emitter. One or more of the selective conductors can be energized to activate the actuator conductors and/or core conductor and cause the actuator conductors and/or core conductor to bend or twist in a preprogrammed manner and return to original shape when deenergized. When de-energized, the actuator conductor and/or core conductor can resume its natural shape.

### CROSS REFERENCE TO RELATED APPLICATIONS

This application is a continuation-in-part of U.S. application Ser. No. 16/269,689, entitled “Programmable Medical Wire System and Method”, filed Feb. 7, 2019, which claims the benefit of U.S. Provisional Application No. 62/628,614, entitled “Programmable Medical Wire System and Method”, filed Feb. 9, 2018, which are incorporated herein by reference.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

### Field of the Invention

The disclosure relates generally to medical equipment and methods of use. More specifically, the disclosure relates to a medical wire assemblies used for access through body passageways or into the body.

### Description of the Related Art

In medical applications, there is often the need to reach portions of the body through the body's own passageways. The body passageways such as the ear, nose, throat, ureter, and rectal passageways allow surgeons and other medical personnel to access different portions of the body that need treatment. Other body passageways include blood vessels.

For example, aneurysms occur generally at a weakened portion of a blood vessel that further stretches the blood vessel walls. One treatment to strengthen the blood vessel walls is to insert a very fine wire into a blood vessel and follow the passageways through the body to the aneurysm. The wire can be followed through an external constantly running CT scan. The medical personnel generally move the wire back and forth. Perhaps twisting the wire one way or the other by trial and error until it hopefully hits the target area. If the wire can form a protective mass or layer inside the aneurysm in time before it burst, then the patient's health and perhaps survival is significantly increased. Thus, success as well as timing in reaching the target area is critical for the patient. Even if the area is reached in time, medical personnel may have difficulties determining the extent of any damage, thickness of any blockages, and severity of a rupture and bleeding. These determinations are critical to the successful treatment of the condition. At present, medical personnel can make assumptions and experience is helpful, but realize the assessment is not based on more complete data that would improve accuracy.

As another example of a need to access remote and sensitive portions of the body, including endovascular access, traumatic brain injury (TBI) affects millions of people per year. TBI is caused by a physical injury to the brain that results in clinically detectable alterations in cognitive processing and function. Injuries can cause a number of conditions including contusions, concussions, penetration, stroke, degenerative diseases, and cancer. TBI gives rise to pathological changes in cerebral physiology, leading to a cascade of secondary injuries ranging from short-term cognitive dysfunction to coma and even death. Preventing secondary injury and neuronal death is challenging for physicians. Reduction in brain tissue oxygenation is one of the hallmark pathophysiological changes that occurs after TBI. Research suggests that oxygen deprivation within the brain after an injury is a leading factor for the onset of such secondary injuries. Evaluating brain tissue oxygenation is of substantial interest among physicians and is monitored in severe cases of TBI. Maintaining normal or elevated oxygen levels can improve treatment outcomes after severe brain trauma. In the brain, oxygen is commonly measured by catheters inserted through the neurovascular system; however, they are limited as they only obtain measurements of a few centimeters of tissue. There is an unmet medical need for new tools and devices that can accurately measure oxygen within the brain to prevent long-term complications.

As yet another example of a need to access remote portions of the body, oxyhemoglobin and deoxyhemoglobin levels are important biomarkers that can be used to measure a wide range of acute to long-term conditions, including neurocognition and tumor growth and control. These levels can be measured by external methods such as pulse oximetry and near infrared spectroscopy (NIRS). Unfortunately, due to light scattering and attenuation in tissue, it is only possible to measure up to a few centimeters of depth in tissue, while many conditions are deeper in the body, such as glioblastoma growth, blood flow to the heart after bypass surgery, or blockages resulting from TBI. There is a need to be able to access deeper into the body.

Other needs for access to remote and sensitive areas of the body including determining thickness including endovascular obstructions, sensing of necrotic tissue such as post-operative evaluations or malignancy regrowth, and other endoscopic medical procedures.

There is a need to produce a medical wire system that can be directed more easily, more reliably, and move quickly through the body passageways, including blood vessels.

### BRIEF SUMMARY OF THE INVENTION

The disclosure provides a system and method for a programmable medical wire system that can be preprogrammed and then controlled and reshaped upon command. The ability to reshape the wire provides the ability of the wire in a body to move quickly, easily, and more successfully reach a target area in the body. In general, the system includes a power supply, a controller, and a multilayered wire assembly.

In at least one embodiment, the programmable medical wire system includes a programmable wire assembly that includes: a core conductor; at least one actuator conductor electrically coupled to the core conductor, the actuator conductor being programmed to move toward a predetermined shape based on actuation; at least one selective conductor electrically coupled to the actuator conductor and configured to be electrically energized to actuate the actuator conductor to move toward the predetermined shape; at least one emitter configured to produce electromagnetic radiation of a given wavelength coupled to the programmable wire assembly; and at least one detector configured to receive emissions of the emitter and communicate signals of the emission remotely from the emitter. One or more of the selective conductors can be energized to activate the actuator conductors and/or core conductor and cause the actuator conductors and/or core conductor to bend or twist in a preprogrammed manner. When the conductors are de-energized, the actuator conductor and/or core conductor can resume its natural shape. By selectively controlling the direction and amount of the bend or twist of one or more of the actuator conductors and/or core conductor, and the time of the bend, the wire assembly can be remotely guided through the body to the target. Other auxiliary equipment, such as micro cameras, cutters, and other equipment, can also be coupled to the wire assembly, and controlled and communicated with through one or more of the selective conductors or other conductors.

In at least a further embodiment, the programmable wire assembly can be inserted in a body passageway or otherwise into the body endoscopically (including for purposes herein endovascular insertion) and one or more emitters of the assembly can operate at a frequency that reflects wave energy from a body portion, such as tissue, proximate to the one or more emitters to determine characteristics of the tissue.

In a still further embodiment, at least two emitters that operate at different frequencies can be used conjunctively to determine comparative characteristics of a body portion, including thickness, oxygen content, and other characteristics. The combination of the medical wire assembly that is steerable with the at least one endoscopic emitter provides access to heretofore difficult areas of a body to reach and analyze proximate tissues and fluids in an endoscopic manner that previously was not available. By endoscopic examination, the accuracy is greatly increased for a more correct diagnosis of the local condition and a more specific and successful diagnoses and operation.

The disclosure also provides a method of using a programmable medical wire system, the method comprising: energizing a selective conductor coupled to an actuator conductor and a core conductor; flowing energy from the selective conductor to the actuator conductor; changing the shape of the actuator conductor in a programmed manner based on an amount of energy provided by the selective conductor; energizing at least one emitter to produce an emission of electromagnetic radiation of a given wavelength; and detecting the emission and communicating signals of the emission remotely from the emitter.

### BRIEF DESCRIPTION OF THE SEVERAL VIEWS OF THE DRAWINGS

FIG. 1 is a schematic perspective view of an example of a programmable medical wire system.

FIG. 2 is a schematic cross sectional view of an example of a selective conductor portion of the programmable medical wire system having a core conductor and selective conductors.

FIG. 3A is a schematic cross sectional view of an example of a transition actuator portion of the programmable medical wire system having a core conductor, actuator conductors electrically coupled to the core conductor, and selective conductors.

FIG. 3B is a schematic cross sectional view of an example of a coupling actuator portion of the programmable medical wire system with a core conductor, actuator conductors electrically coupled to the core conductor, and selective conductors electrically coupled to the actuator conductors.

FIG. 4 is a schematic cross sectional view of an example of an equipment portion of the programmable medical wire system having a core conductor, a twist conductor electrically coupled to a core conductor, and at least one selective conductor electrically coupled to the twist conductor.

FIG. 5 is a schematic diagram of the programmable medical wire assembly with various examples of cross sections of the assembly at the noted locations.

FIG. 6 is a schematic view of an enlarged equipment portion of a programmable wire assembly endoscopically inserted into a body with multiple emitters and multiple detectors.

FIG. 7 is a schematic view of a similar embodiment as in FIG. 6 that is used in a thickness application.

FIG. 8 is a schematic view of a similar embodiment as in FIG. 6 that is used in a necrotic application.

FIG. 9 is a schematic view of an enlarged equipment portion of a programmable wire assembly endoscopically inserted into a body.

FIG. 10 is a schematic view of another embodiment of the programmable wire assembly with another exemplary type of equipment as an end effector.

FIG. 11 is a schematic view of an enlarged equipment portion of a programmable wire assembly endoscopically inserted into a body with a detector on the equipment portion.

### DETAILED DESCRIPTION

The Figures described above and the written description of specific structures and functions below are not presented to limit the scope of what Applicant has invented or the scope of the appended claims. Rather, the Figures and written description are provided to teach any person skilled in the art how to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present disclosure will require numerous implementation-specific decisions to achieve the developer's ultimate goal for the commercial embodiment. Such implementation-specific decisions may include, and likely are not limited to, compliance with system-related, business-related, government-related, and other constraints, which may vary by specific implementation, location, or with time. While a developer's efforts might be complex and time-consuming in an absolute sense, such efforts would be, nevertheless, a routine undertaking for those of ordinary skill in this art having benefit of this disclosure. It must be understood that the inventions disclosed and taught herein are susceptible to numerous and various modifications and alternative forms. The use of a singular term, such as, but not limited to, “a,” is not intended as limiting of the number of items. Further, the various methods and embodiments of the system can be included in combination with each other to produce variations of the disclosed methods and embodiments. Discussion of singular elements can include plural elements and vice-versa. References to at least one item may include one or more items. Also, various aspects of the embodiments could be used in conjunction with each other to accomplish the understood goals of the disclosure. Unless the context requires otherwise, the term “comprise” or variations such as “comprises” or “comprising,” should be understood to imply the inclusion of at least the stated element or step or group of elements or steps or equivalents thereof, and not the exclusion of a greater numerical quantity or any other element or step or group of elements or steps or equivalents thereof. The device or system may be used in a number of directions and orientations. The terms “top”, “up’, “upward’, “bottom”, “down”, “downwardly”, and like directional terms are used to indicate the direction relative to the figures and their illustrated orientation and are not absolute relative to a fixed datum such as the earth in commercial use. The term “inner,” “inward,” “internal” or like terms refers to a direction facing toward a center portion of an assembly or component, such as longitudinal centerline of the assembly or component, and the term “outer,” “outward,” “external” or like terms refers to a direction facing away from the center portion of an assembly or component. The term “coupled,” “coupling,” “coupler,” and like terms are used broadly herein and may include any method or device for securing, binding, bonding, fastening, attaching, joining, inserting therein, forming thereon or therein, communicating, or otherwise associating, for example, mechanically, magnetically, electrically, chemically, operably, directly or indirectly with intermediate elements, one or more pieces of members together and may further include without limitation integrally forming one functional member with another in a unitary fashion. The coupling may occur in any direction, including rotationally. The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions. Some elements are nominated by a device name for simplicity and would be understood to include a system of related components that are known to those with ordinary skill in the art and may not be specifically described. Some elements are described with a given element number and where helpful to describe embodiments with various examples are provided in the description and figures that perform various functions and are non-limiting in shape, size, description, but serve as illustrative structures that can be varied as would be known to one with ordinary skill in the art given the teachings contained herein. Element numbers with suffix letters, such as “A”, “B”, and so forth, are to designate different elements within a group of like elements having a similar structure or function, and corresponding element numbers without the letters are to generally refer to one or more of the like elements.

In at least one embodiment, the programmable medical wire system includes a programmable wire assembly that includes: a core conductor; at least one actuator conductor electrically coupled to the core conductor, the actuator conductor being programmed to move toward a predetermined shape based on actuation; at least one selective conductor electrically coupled to the actuator conductor and configured to be electrically energized to actuate the actuator conductor to move toward the predetermined shape; at least one emitter configured to produce electromagnetic radiation of a given wavelength coupled to the programmable wire assembly; and at least one detector configured to receive emissions of the emitter and communicate signals of the emission remotely from the emitter. One or more of the selective conductors can be energized to activate the actuator conductors and/or core conductor and cause the actuator conductors and/or core conductor to bend or twist in a preprogrammed manner. When the conductors are de-energized, the actuator conductor and/or core conductor can resume its natural shape. By selectively controlling the direction and amount of the bend or twist of one or more of the actuator conductors and/or core conductor, and the time of the bend, the wire assembly can be remotely guided through the body to the target. Other auxiliary equipment, such as micro cameras, cutters, and other equipment, can also be coupled to the wire assembly, and controlled and communicated with through one or more of the selective conductors or other conductors.

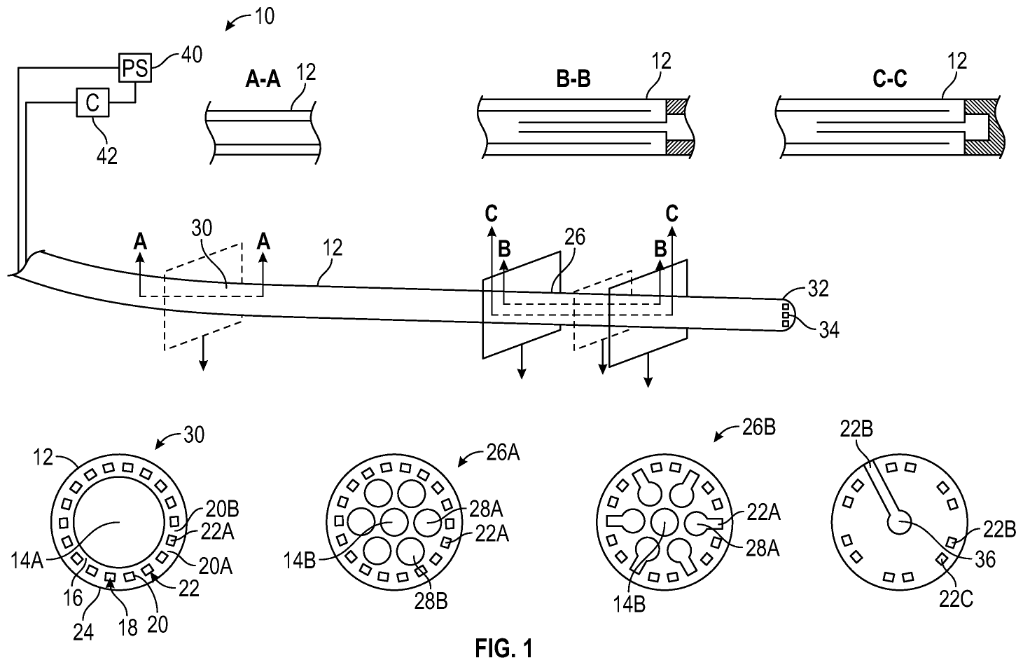

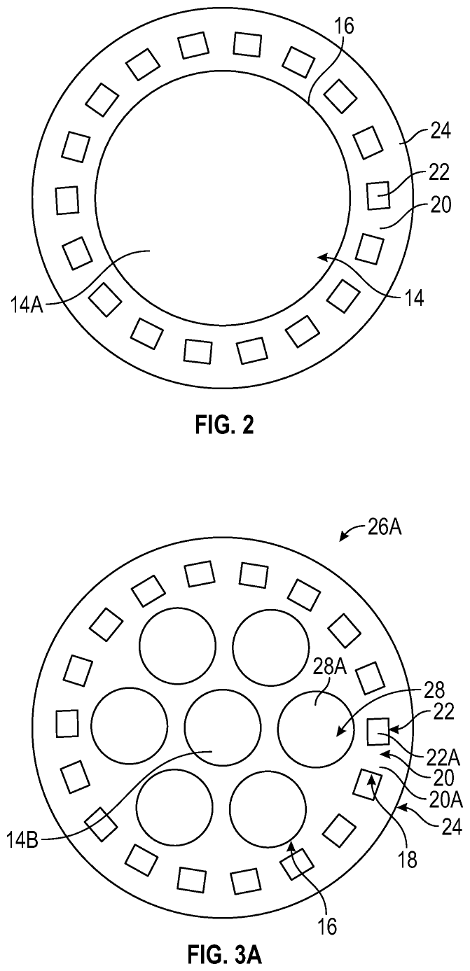

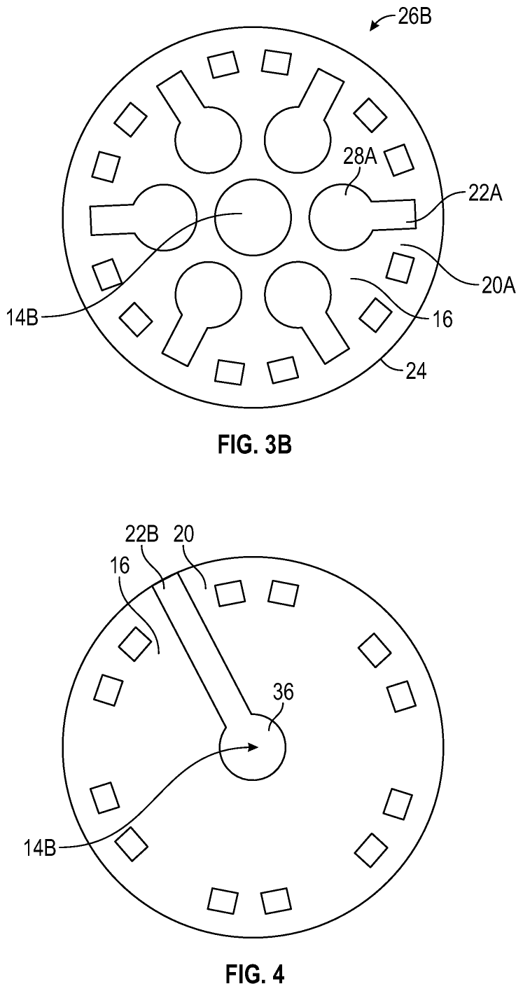

FIG. 1 is a schematic perspective view of an example of a programmable medical wire system. FIG. 2 is a schematic cross sectional view of an example of a selective conductor portion of the programmable medical wire system having a core conductor and selective conductors. FIG. 3A is a schematic cross sectional view of an example of a transition actuator portion of the programmable medical wire system having a core conductor, actuator conductors electrically coupled to the core conductor, and selective conductors. FIG. 3B is a schematic cross sectional view of an example of a coupling actuator portion of the programmable medical wire system with a core conductor, actuator conductors electrically coupled to the core conductor, and selective conductors electrically coupled to the actuator conductors. FIG. 4 is a schematic cross sectional view of an example of an equipment portion of the programmable medical wire system having a core conductor, a twist conductor electrically coupled to a core conductor, and at least one selective conductor electrically coupled to the twist conductor.

The programmable medical wire system 10 generally includes a multilayered programmable wire assembly 12 having a variety of conductors described herein, a power supply 40 for energizing conductors in the wire assembly, and a controller 42 for controlling which conductors are energized. The term “wire” is used broadly herein, and includes single strand and multistrand wires as well as wires formed by deposition of conductive materials and other methods. The material of the “wire” can be various conductive materials, including metals, semi-metals, conductive metal oxides, and other conductive materials. In general, the wires are flexible to accommodate an amount of bending, twisting, and other movement suitable for the application. In general, the programmable wire assembly 12 is an elongated multilayer assembly having one or more portions that are preprogrammed to move laterally or rotationally (or “twist”) relative to a main body of the wire assembly. In at least one embodiment, the movement can be preprogrammed by pre-setting an actuator conductor, a core conductor, or a combination thereof to move in a given manner when energized and return to a normal shape when de-energized.

In at least one embodiment, at least one selective conductor portion 30 (such as the selective conductor portion 30A shown in FIG. 1) of the wire assembly 12 is formed by a core conductor 14 with a series of layers surrounding the core conductor. The core conductor may vary in size and configuration along the wire assembly 12. The insert in FIG. 1 and the larger image in FIG. 2 show a cross section of the selective conductor portion 30A in more detail. An insulation layer 16 can be formed around the core conductor. A selective conductor layer 18 can be formed around the insulation layer 16. The selective conductor layer 18 can be radially divided into insulation portions 20 that separate selective conductors 22. For example, selective conductor 22A is bounded by an insulation portion 20A on one side and insulation portion 20B on a distal side. A further layer 24 can be formed over the selective conductor layer 18 to protect the wire assembly as a shield from external fluids and other materials. Therefore, advantageously, the layer 24 can be biocompatible and flexible to allow movement through the body passageways. One example of a suitable material is a chemical vapor deposition polymer.

In at least one embodiment, at least one actuator portion 26A (generally “26”) can be formed in the programmable wire assembly 12. The actuator portion 26 can be electrically coupled with the selective conductor portion 30. In at least one embodiment, the actuator portion 26 can be formed for illustrative purposes from a transition actuator portion 261A and a coupling actuator portion 262A.

The insert in FIG. 1 and FIG. 3A shows details of the transition actuator portion 261A. The transition actuator portion 261A can include a smaller diameter core conductor 14 that is surrounded by insulation, similar to the insulation layer 16. The insulation insulates a plurality of actuator conductors 28A, 28B, and so forth (generally “28”) from the core conductor 14. The actuator conductors 28 are electrically coupled to the core conductor 14 generally on a proximal end toward the selective conductor portion 30A and generally not on the distal end. Further, the actuator conductors 28 are insulated from each other to allow independent control of the actuator conductors 28 through electrical energy provided by selection of the correspondingly selective conductor(s) 22. The selective conductor layer 18 with the selective conductors 22 remains formed around the core conductor 14 as well as now the actuator conductors 28.

The actuator conductors 28 can be made of materials that change shape with temperature changes. For example and without limitation, a suitable material can be a “shaped memory effect” (“SME”) material, such as nickel-titanium (nitinol). Such a material responds to heat such as through electrical stimulation through a resistive material and can return to a natural state when the heat is removed. The chemical composition can change the amount of heat that is required to produce a given movement. To program the shape, the material can be heated to a certain level, bent or twisted or otherwise formed to a desired shape, and then cooled at that shape to set the shape. The shape will resume with suitable heat. Thus, the material forms a “memory” of a heated shape.

The desired shape can thus be programmed in manufacturing of the programmable wire assembly 12. With resistive electrical energy resulting in heat in the actuator portion 26, the programmable wire assembly can move in a variety of directions depending on which actuator conductor 28 is heated, such as through electrical current.

The coupling actuator portion 262A shown in the insert in FIG. 1 and a larger view in FIG. 3B is similarly constructed as in transition actuator portion 261A. However, the actuator conductors 28 are electrically coupled to corresponding selective conductors 22. For example, actuator conductor 28A can be electrically coupled to a selective conductor 22A. The coupling is surrounded by the insulation from insulation layer 16 and the insulation 20. Essentially, the insulation layer 16 has been bridged by coupling the actuator conductor with the selective conductor. Thus, when the actuator conductor 28A is desired to be actuated, electrical current can flow through the selective conductor 22A and heat up the actuator conductor 28A. The heat can cause the actuator conductor 28A to move in a preprogrammed manner depending on the level of heat or other energy created by the actuator conductor 28A. In a similar fashion, other selective conductors 22 can be electrically coupled with corresponding actuator conductors 28. Thus, selective actuation of one or more of the actuator conductors can cause the programmable wire assembly to move in a variety of directions. In similar manner, the core conductor can be electrically coupled with a selective conductor to flow electrical current into the core conductor to cause the core conductor to move in a preprogrammed manner if the core conductor has been preset to do so upon activation.

An equipment portion 32 of the programmable wire assembly 12 can provide further flexibility and use of the programmable wire assembly 12. In at least one embodiment, an end of the core conductor 14 can be pre-programmed into a twisted shape to form a portion as a twist conductor 36. One or more twist conductors (not shown) can be used in different locations that are coupled to the core conductor 14. The twist conductor 36 can be electrically coupled to a selective conductor 22B in similar fashion as has been described. Upon actuation of the selective conductor 22B to create heat on the twist conductor 36, the twist conductor 36 can twist along a longitudinal axis of the twist conductor (that is, along a length of the twist conductor) and rotate the programmable wire assembly depending on the amount of heat provided. In this embodiment, the twist conductor can rotate when activated the equipment portion 32. While the equipment portion 32 is used for illustration, a twist conductor 36 can be inserted at other portions along the length of the programmable wire assembly 12.

The equipment portion 32 can also be used to support equipment 34 for various functions. For example and without limitation, such equipment can include cameras, sensors, cutters, and other tools. Such equipment may be micro-sized or otherwise sized as appropriate. The equipment can be controlled and communicated with through one or more of the selective conductors.

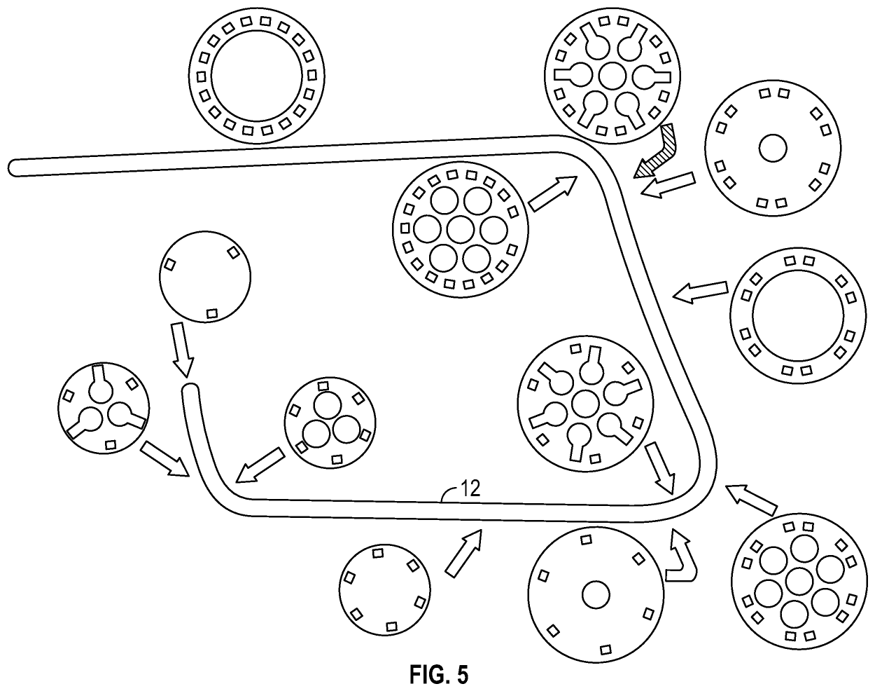

FIG. 5 is a schematic diagram of the programmable medical wire assembly with various examples of cross sections of the assembly at the indicated locations. In some embodiments, the programmable work assembly 12 can include a plurality of selective conductor portions 30 and actuator portions 26. The actuator portions 26 can be selectively controlled by coupling different selective conductors 22 with actuator conductors 28 in each of the actuator portions 26.

In this example, the programmable work assembly 12 includes a first selective conductor portion that includes the selective conductors 22 and the core conductor 14 described above, followed by a first actuator portion 26A having the transition actuator portion 261A and coupling actuator portion 262A, as described above.

A second selective conductor portion 30B can follow the first actuator portion 26A along the programmable wire assembly 12 and is illustrated with a portion 301B and a portion 302B. A first transition conductor guide portion 301B shows the remaining selective conductors 22 that are available for subsequent control downstream without the actuator conductors 28 and selective conductors 22 that were used in the first actuator portion 26A. The first transition conductor guide portion 301B also shows the core conductor 14 that was used in the first actuator portion 26A. A second transition conductor guide portion 302B still shows the remaining selective conductors, but with a relatively enlarged core conductor 14 that can be more readily coupled to additional actuator conductors 28 in the next downstream portion.

A second actuator portion 26B can follow the second selective conductor portion 30B along the programmable wire assembly 12. The second actuator portion 26B can include a transition actuator portion 261B and coupling actuator portion 262B. The transition actuator portion 261B includes the core conductor 14 with actuator conductors 28 coupled to the core conductor and remaining selective conductors 22. The coupling actuator portion 262B is similarly constructed as the transition actuator portion 261B, but with the actuator conductors 28 also coupled to one or more of the remaining selective conductors 22. One or more of the selective conductors can be energized to actuate their corresponding actuator conductors 28 to move in one or more directions as described above.

A third selective conductor portion 30C can follow the second actuator portion 26B along the programmable wire assembly 12 and is illustrated with a portion 301C and a portion 302C. A first transition conductor guide portion 301C shows the remaining selective conductors 22 that are available for subsequent control downstream without the actuator conductors 28 and selective conductors 22 that were used in the second actuator portion 26B. The first transition conductor guide portion 301C also shows the core conductor 14 that was used in the second actuator portion 26B. A second transition conductor guide portion 302C still shows the remaining selective conductors 22, but with a relatively enlarged core conductor 14 that can be more readily coupled to additional actuator conductors 28 in the next downstream portion. The second transition conductor guide portion 302C (and others portions) also illustrates that the peripheral dimension can be reduced as appropriate as the number of selective conductors 22 and/or actuator conductors 28 is reduced in the programmable wire assembly 12.

A third actuator portion 26C can follow the third selective conductor portion 30C along the programmable wire assembly 12. The third actuator portion 26C can include a transition actuator portion 261C and coupling actuator portion 262C. The transition actuator portion 261C includes actuator conductors 28 coupled to the core conductor 14 of the third selective conductor portion 30C and remaining selective conductors 22. The coupling actuator portion 262C is similarly constructed as the transition actuator portion 261C, but with the actuator conductors 28 also coupled to one or more of the remaining selective conductors 22. One or more of the selective conductors can be energized to actuate their corresponding actuator conductors 28 to move in one or more directions as described above.

An equipment portion 32 of the programmable wire assembly 12 can provide further flexibility and use of the programmable wire assembly 12. The equipment portion 32 can also be used to support and communicate with equipment for various functions, as described above. Such equipment may be micro-sized or otherwise sized as appropriate. The equipment can be controlled and communicated with through one or more of the selective conductors 22.

A wire assembly that can be intelligently guided through various openings in an animal or human body (more generally an “object”), or otherwise to enter and be guided in the body can be advantageously used in multiple ways. In general, such a wire assembly can be used to determine characteristics or other conditions of the body or body portions or to take action on or in the body or body portions. As non-limiting examples, the wire assembly can be used for oxygen sensing in the blood as an oximeter or sensing other biomarkers such a lactic acid and tumor markers, determining tissue thickness including endovascular obstructions that may be difficult to detect accurately with other known methods, sensing of necrotic tissue such as post-operative evaluations or malignancy regrowth, resection of tissues including tumors, collecting tissue samples, cutting through cellular and vascular blockages, delivery of a payload such as nanoparticles that can be activated by the wire assembly or by a separate device, endovascular embolisms, cranial surgeries with minimum tissue disruption, and other endoscopic medical procedures, including for purposes herein endovascular medical procedures. Further, the wire assembly can be guided robotically from a remote location for increased availability of intricate medical procedures under adverse conditions such as in the armed forces.

In more detail for least a category of the examples, one or more lights can be included with the wire assembly and located for example on a leading portion of the wire assembly into the body. The light or lights can be of one or more frequencies, including visible, near-infrared (NIR), infrared (IR), ultraviolet (UV), or other frequencies. Multiple lights may have different frequencies that are absorbed differently by the body where the difference in absorption can indicate a biomarker value of interest.

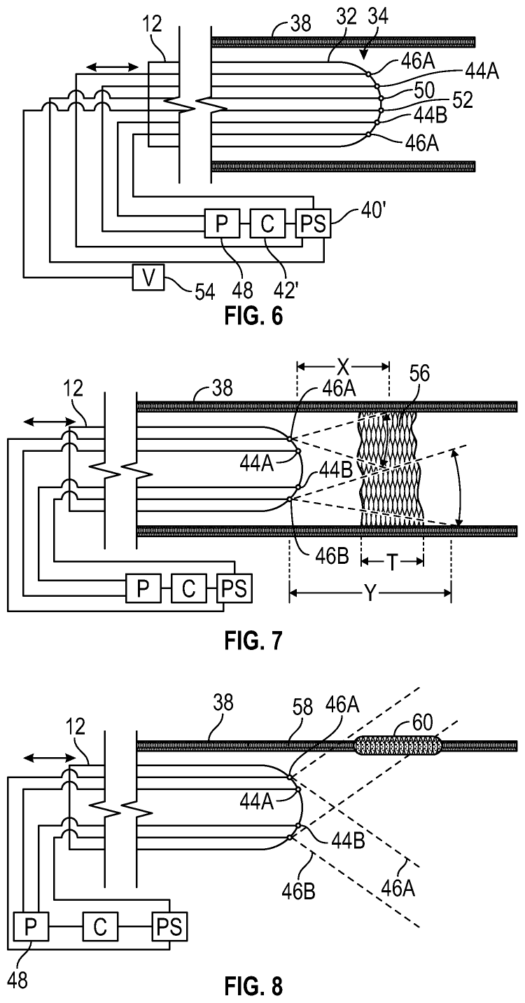

FIG. 6 is a schematic view of an enlarged equipment portion of a programmable wire assembly endoscopically inserted into a body with multiple emitters and multiple detectors. The programmable wire assembly 12 is selectively bendable for navigating through the body, such as a vascular system to areas of interest. The size can vary for the application and for many commercially viable applications with the greatest need, the diameter can be, for example, 100-250 micrometers (μm) or less, including 100-150 μm in diameter. The programmable wire assembly can contain shape memory alloy, such as Nitinol. Some biomarkers, such as oxygen or lack thereof, and other parameters can be measured by comparison of results from different wavelengths from emitters.

In this embodiment, the emitters and detectors can be coupled to the equipment portion 32. For an embodiment where the detector can be so located, the embodiment advantageously does not need to transmit a signal through the intervening tissues or other structure of the body, such as bones or skull, to an external detector. Thus, the programmable wire assembly can be inserted deeper into the body for an increased penetration and provide a higher sensitivity for measurements. The equipment 34 can include an emitter 46A and emitter 46B that can produce electromagnetic waves, such as a light, including from a light emitting diode (LED). The equipment 34 can be mounted on an end of the programmable wire assembly as shown or some other location along the length of the assembly as may be preferable for an application. The emitter 46A and emitter 46B can be coupled to a power supply 40′, which can be a separate power supply or integrated with the power supply 40 described herein. The equipment 34 can include a detector 44A and detector 44B for emitter 46A and emitter 46B, respectively, and be also be coupled to the power supply 40′ and a controller 42′. In other embodiments, the detector 44A and/or detector 44B can be external to the tissue or even external to the body and coupled to a controller 42′ and the power supply 40′, similar to an external embodiment shown in FIG. 10A. The controller 42′ can also be coupled with the power supply 40′ to control power to the emitter 46A and emitter 46B. The controller 42′ can be a separate controller or integrated with the controller 42 described herein. The emitter 46A and emitter 46B can be configured to produce appropriate wavelengths suitable for the purpose. For example, for the measurement of oxygen, an infrared (IR) wavelength, including a near infrared (NIR) wavelength, is generally suitable. An exemplary range for the lights frequencies is from 620 nm-1000 nm, where one frequency could be in the 700's nm and the other frequency in the 900's nm, although the ultimate value can vary outside of the range. Extending to the wavelength to a mid-infrared region, for example 3-5 μm or longer, can obtain information where molecules can have higher sensitivities with their vibrational and rotational resonance frequencies, enabling measurements of smaller amounts. For example, the emitters can have combinations of red/green, blue/purple, and others that have different depth penetrations through tissue or other structure. The emitters 46A and 46B are illustrated as separate emitters, but functionally a dual wavelength emitter is included within the scope of emitters 46A and 46B. Similarly, the detectors 44A and 44B are illustrated as separate detectors, but functionally a dual wavelength detector is included within the scope of detectors 44A and 44B. Further, more than two emitters and/or detectors can be used redundancy, error checking, increased corroboration, and other purposes.

The emitter 46A and emitter 46B can be powered to produce emissions toward the tissue at the desired frequencies. The emitter 46A can produce a wavelength A and detector 44A can receive an emission affected by the tissue that is generally reflected, refracted, or otherwise changed from the emitter 46A. The emitter 46B can produce a wavelength B, generally at a different wavelength that emitter 46A, and detector 44B can receive such emission likewise affected by the tissue that is generally reflected, refracted, or otherwise changed from the emitter 46B. The detectors can provide signals representing such emissions to the processor 48 to process the information and determine the oxygen content. The processor 48 can provide information to the controller 42′ to guide the controller on controlling the emitters and/or the detectors.

The different wavelengths can be used to determine various biomarkers. As an example, the different wavelengths can be used to determine oxyhemoglobin and deoxyhemoglobin levels. The ability to go deep into the body especially with the detector provides a small, biocompatible addressable system that can be used to transmit or receive infrared light and transmit the resulting data. The ability to implant or insert infrared emitters has the potential to improve measurement sensitivity by reducing the scattering path length by at least a factor of two. This capability may provide increased potential for long-term care of recovering patients in definitive care facilities, as well as compact, robust, easier-to-use systems for patient stabilization and improved monitoring of key biomarkers at emergency and urgent care centers. These systems even could be utilized during patient transport, for example, to verify that oxygenation levels in the brain are sufficient.

Further, the embodiment (and other embodiments) can include a visible light source 50 that can be powered by the power supply 40′ and can be controlled by the controller 42′. A sensor 52 coupled at an appropriate position, such as the equipment portion 32, can sense the light and send an external signal to a viewer 54, such as a camera, optical lens, or other equipment, to provide optical guidance to medical staff.

FIG. 7 is a schematic view of a similar embodiment as in FIG. 6 that is used in a thickness application. A thickness T of a tissue 56 in the body 38, such as an obstruction in the colon and blood vessels or tissue that has been compromised from disease or injury can be important to know for deciding medical options in treatment. While different methods are typically used (including radiographic dyes with X-ray or scanning equipment and MRIs), it is sometimes difficult to determine accurately the thickness T of the tissue. Navigating one or more of the embodiments described herein in proximity to the tissue can yield results that are more accurate. Multiple emitters 46A and 46B operating at different wavelengths can provide different depths of penetration in or through the tissue or other structure. One wavelength can be shorter to penetrate only partially into the tissue at a distance X and the other wavelength can extend further or even past the thickness at a distance Y. A comparison of the results can indicate a thickness and potentially other characteristics.

FIG. 8 is a schematic view of a similar embodiment as in FIG. 6 that is used in a necrotic application. Necrotic tissue 60, such as from tumors, that is dying or dead can be determined from emitters emitting at different frequencies and comparing readings of tissue. Using the illustrated embodiment, the emitter 46A can produce an emission at its wavelength into healthy tissue 58 and into the necrotic tissue 60 at a different location if necessary. Likewise, the emitter 46B operating at its wavelength, which is different than emitter 46A, can emit into healthy tissue and into the necrotic tissue at a different location if necessary. The detectors 44A and 44B can detect the resulting transmission from the emitters 46A and 46B into or through the tissue, respectively, and return the signals to the processor 48, as has been described above. The processor can determine the existence of the necrotic tissue and can determine the location and size of the necrotic tissue.

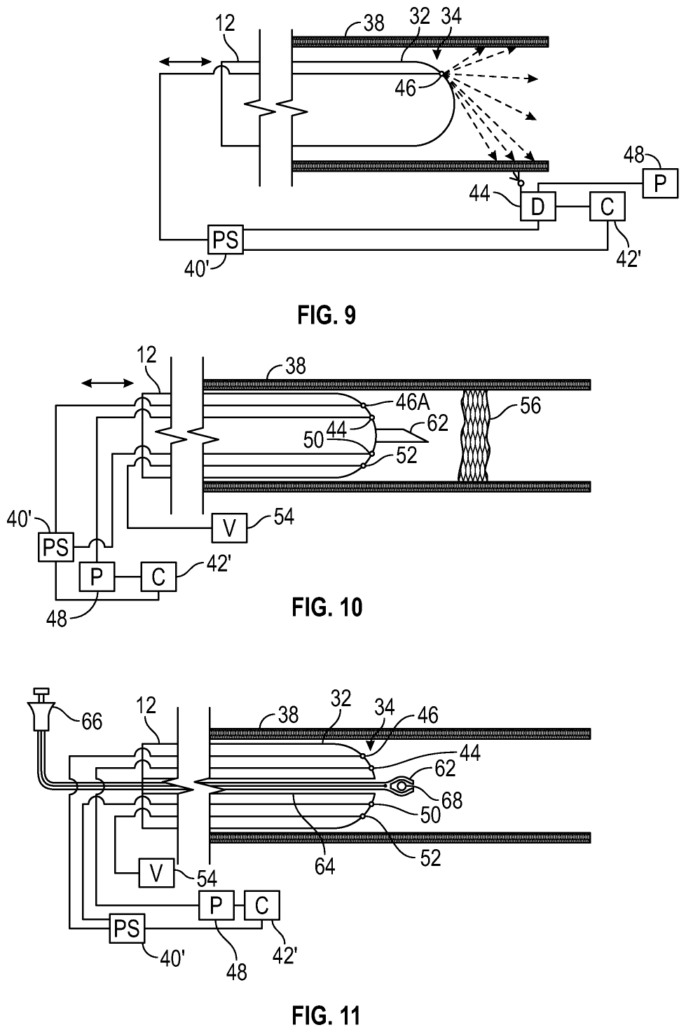

FIG. 9 is a schematic view of an enlarged equipment portion of a programmable wire assembly endoscopically inserted into a body. In this embodiment, the equipment 34 can include an emitter 46 that can produce electromagnetic waves, such as a light, including a light emitting diode (LED), ultrasonic waves, or other wavelengths. The embodiment is similar to prior embodiments with the controller, power supply, and other components using two or more emitters and detectors, but where the difference in wavelength emission from multiple emitters into tissue or other structure for measurement may not be needed. The equipment 34 can be mounted on an end of the programmable wire assembly as shown or some other location along the length of the assembly as may be preferable for an application. The emitter 46 can be coupled to a power supply 40′, which can be a separate power supply or integrated with the power supply 40 described herein. A detector 44 can be external to the tissue or even external to the body and coupled to a controller 42′ and the power supply 40′. Alternatively, the detector 44 can also be coupled to the equipment portion 34 of the programmable wire assembly and inserted into the body with the emitter 46, similar to the embodiment shown in FIG. 6. The controller 42′ can be a separate controller or integrated with the controller 42 described herein. The controller 42′ can also be coupled with the power supply 40′ to control power to the emitter 46. The emitter 46 can be configured to produce any wavelength suitable for the purpose. The emitter 46 can be powered to produce the emission at the desired frequency where the detector 44 can receive the emission. The detector can provide signals representing such emission to the processor 48 to process the information. The processor 48 can provide information to the controller 42′ to guide the controller on controlling the emitter and/or the detector. For example, the equipment 34 can include an ultrasonic sensor. The ultrasonic sensor can be used to determine the position of the programmable wire assembly 12 and therefore a direction of travel of the assembly. Using that information, medical personnel can more readily guide the assembly as the assembly is pushed toward a targeted area.

FIG. 10 is a schematic view of another embodiment of the programmable wire assembly with another exemplary type of equipment as an end effector. The equipment 34 can include an end effector 62 illustrated as cutter, such as a surgical knife or laser for cutting a structure 56, such as a tissue or a blockage. The end effector 62 can be coupled to an end or other appropriation location on the programmable wire assembly and can be advanced toward the structure 56 to be cut or punctured. In an alternative embodiment, the programmable wire assembly can have a channel through which the end effector can slide to advance from the channel and retract into the channel, such as shown in FIG. 11. The programmable wire assembly 12 can also include a visible light source 50 and a light sensor 52 as described in FIG. 6 that can sense reflections of the light on objects and send a signal to a viewer 54 to assist medical personnel with the analysis or procedure. The equipment 34 can be coupled with a power supply 48, a processor 40′ and a controller 42′ as described above. The end effector 62 as a cutter is illustrative and can include other types of equipment such as a laser, cauterizing equipment, ultrasonic sensors, and so forth that is pertinent to a given application.

Further, an end effector can be coupled to other embodiments disclosed herein, including but limited to the embodiments having two emitters. For example, the two emitters could identify a type of endovascular structure, such as a tissue or blockage, or thickness of structure. The effector could perform a medical procedure on the structure.

FIG. 11 is a schematic view of an enlarged equipment portion of a programmable wire assembly endoscopically inserted into a body with a detector on the equipment portion. Similar to other embodiments, equipment 34 coupled to an end or other appropriate location of the programmable wire assembly 12, wherein the equipment can include an emitter 46 and detector 44, a visible light source 50 and a light sensor 52 with a viewer 54. The equipment 34 can be coupled to a power supply 40′, controller 42′, and processor 48. The embodiment can further include a channel 64. An extender 66 can extend an end effector 62 through the channel. The end effector 62 can hold a payload 68 to be delivered to an appropriate site. The payload 68 can be nanoparticles for treatment or other benefits, medicine, or other deliverables. In at least one embodiment, the payload can be a substance to embolize a vessel or other bodily conduit. An example of a substance can be a bio-compatible glue that can stiffen in place to resist flow through the conduit. If the glue needs activation by an energy source, the emitter 46 could be configured to provide appropriate energy, such as ultraviolet or infrared light or other energy. An example could be embolizing a vessel that has been irreparably damaged or one that is providing a blood supply to a tumor. In some embodiments, the emitter can be a laser such as through an optical fiber to cut or cauterize a tissue.

As another embodiment, the end effector 62 can be a resector configured to slide in the channel and extend to a body portion to be resected. The wire assembly 12 can be guided to an appropriate location and the resector extended to resect a blockage, growth, or other structure. For example, the wire assembly could be guided to a vessel providing blood to a tumor. The resector could resect the tumor from the inside the vessel, piece by piece, through the channel 64. The wire assembly could further extend through the vessel if needed to perform the medical procedure, and after withdrawing back into the vessel embolize the perforation in the vessel. The embodiment is representative and the channel and effectors can be included with any of the embodiment disclosed herein and other embodiments.

It should be noted that several of the embodiments illustrate the programmable wire assembly inside a body passage, as in endovascular procedures. However, the programmable wire assembly is not so limited, as described above, and includes more general endoscopic applications in the body outside of a vascular system and other body passages, such as between organs, interstitially, and in other areas where a remote access is useful.

The invention has been described in the context of preferred and other embodiments and not every embodiment of the invention has been described. Obvious modifications include variations in the number of components that may be combined, the number of layers and/or, conductors, shapes, and purposes, various end effectors, location of the equipment portion and equipment along the programmable wire assembly and other variations and associated methods of use and manufacture that an ordinary person skilled in the art would envision given the teachings herein. The disclosed and undisclosed embodiments are not intended to limit or restrict the scope or applicability of the invention conceived of by the Applicant, but rather, in conformity with the patent laws, Applicant intends to protect fully all such modifications and improvements that come within the scope of the following claims.

What is claimed is:

1. A programmable medical wire system, comprising:
a programmable wire assembly, comprising:
a core conductor;
at least one actuator conductor electrically coupled to the core conductor, the actuator conductor being programmed to move toward a predetermined shape based on actuation;
at least one selective conductor electrically coupled to the actuator conductor and configured to be electrically energized to actuate the actuator conductor to move toward the predetermined shape;
at least one emitter configured to produce electromagnetic radiation of a given wavelength coupled to the programmable wire assembly; and
at least one detector configured to receive emissions of the emitter and communicate signals of the emission remotely from the emitter.

2. The system of claim 1, wherein the emitter wavelength is an infrared, near infrared, or ultrasonic wavelength.

3. The system of claim 1, further comprising a first emitter configured to produce an emission at a first wavelength and a second emitter configured to produce an emission at a second wavelength different than the first wavelength.

4. The system of claim 3, wherein a characteristic of a body can be determined by emission results at the first wavelength compared to emission results at the second wavelength.

5. The system of claim 4, wherein the characteristic of the body comprises an oxygen content.

6. The system of claim 4, wherein the characteristic of the body comprises an existence of necrotic tissue.

7. The system of claim 1, wherein the programmable wire assembly further comprises an end effector configured to alter a condition of a body that is engaged by the end effector.

8. The system of claim 7, wherein the end effector comprises at least one of a cutter, laser, resector, and ultrasonic emitter.

9. The system of claim 1, further comprising a channel formed through the programmable wire assembly, and wherein the programmable wire assembly further comprises an end effector configured to deliver a payload through the channel.

10. The system of 9, wherein the payload is activated by the electromagnetic radiation from the at least one emitter.

11. The system of claim 1, further comprising a channel formed through the programmable wire assembly, and wherein the programmable wire assembly further comprises an end effector configured as a slidable resector through the channel to resect a body portion.

12. The system of claim 1, wherein the core conductor is programmed to move toward a predetermined shape based on actuation by a selective conductor.

13. The system of claim 1, wherein the programmable wire assembly comprises a peripheral layer of a plurality of selective conductors surrounding a layer of a plurality of actuator conductors that are selectively insulated from the surrounding the core conductor, wherein two or more of the actuator conductors are programmed to bend toward the predetermined shape.

14. The system of claim 1, wherein the programmable wire assembly comprises at least one selective conductor portion comprising a plurality of the selective conductors and the core conductor.

15. The system of claim 14, wherein the programmable wire assembly comprises at least one actuator portion comprising a plurality of the actuator conductors electrically coupled with the plurality of the selective conductors.

16. The system of claim 1, wherein the programmable wire assembly comprises at least one data portion comprising one or more attachments electrically coupled to one or more selective conductors and configured to obtain medical data from use of the programmable wire assembly.

17. The system of claim 1, wherein at least one of the actuator conductors comprises a twist conductor configured to twist along a longitudinal axis of the twist conductor.

18. The system of claim 1, wherein the programmable wire assembly comprises
a first selective conductor portion comprising a first set of selective conductors and a second set of selective conductors;
a first actuator portion comprising a first set of actuator conductors electrically coupled with the first set of selective conductors;
a second selective conductor portion comprising the second set of selective conductors; and
a second actuator portion comprising a second set of actuator conductors electrically coupled with the second set of selective conductors.

19. The system of claim 18, wherein the second set of selective conductors is insulated from the first set of selective conductors in the first selective conductor portion.

20. The system of claim 18, wherein the second actuator portion is disposed at a different longitudinal position along the programmable wire assembly than the first actuator portion and configured to control movement of the programmable wire assembly independently of the first actuator portion.

21. A method of using a programmable medical wire system, the method comprising:
energizing a selective conductor coupled to an actuator conductor and a core conductor;
flowing energy from the selective conductor to the actuator conductor;
changing the shape of the actuator conductor in a programmed manner based on an amount of energy provided by the selective conductor;
energizing at least one emitter to produce an emission of electromagnetic radiation of a given wavelength; and
detecting the emission and communicating signals of the emission remotely from the emitter.

22. The method of claim 21, wherein energizing the at least one emitter to produce the emission comprises emitting an infrared, near infrared, or ultrasonic wavelength.

23. The method of claim 21, wherein energizing the at least one emitter to produce the emission comprises energizing a first emitter to produce an emission at a first wavelength and energizing a second emitter to produce an emission at a second wavelength different than the first wavelength.

24. The method of claim 23, further comprising determining a characteristic of a body by comparing emission results at the first wavelength compared to emission results at the second wavelength.

25. The method of claim 24, further comprising determining a characteristic of a body comprises determining an oxygen content.

26. The method of claim 24, further comprising determining a characteristic of a body comprises determining existence of necrotic tissue.

27. The method of claim 21, further comprising using an end effector coupled on the programmable wire assembly to alter a condition of a body.

28. The method of claim 27, wherein using the end effector comprising at least one of cutting, applying a laser, resecting, and emitting an ultrasonic frequency directed toward a body.

29. The method of claim 21, further comprising a channel formed through the programmable wire assembly, and wherein the method further comprises delivering a payload through the channel.

30. The method of claim 29, further comprising activating the payload by emitting electromagnetic radiation toward the payload.

31. The method of claim 21, further comprising a channel formed through the programmable wire assembly, and wherein the method comprises resecting a body portion through the channel.

32. The method of claim 21, wherein the programmable medical wire system comprises a first selective conductor portion comprising a first set of selective conductors and a second set of selective conductors; a first actuator portion comprising a first set of actuator conductors electrically coupled with the first set of selective conductors; a second selective conductor portion comprising the second set of selective conductors; and a second actuator portion comprising a second set of actuator conductors electrically coupled with the second set of selective conductors, and the method further comprises:
energizing a selective conductor of the first set of selective conductors to actuator an actuator conductor of the first set of actuator conductors in the first actuator portion to change the shape of the actuator conductor in a programmed manner based on an amount of energy provided by the selective conductor; and
energizing a selective conductor of the second set of selective conductors to actuator an actuator conductor of the second set of actuator conductors in the second actuator portion to change the shape of the actuator conductor in a programmed manner based on an amount of energy provided by the selective conductor in the second set of selective conductors; and
controlling movement of the programmable wire assembly in the first actuator portion independently of controlling movement of the programmable wire assembly in the second actuator portion.

33. The method of claim 21, wherein the actuator conductor comprises a twist conductor and the method further comprises:
energizing a selective conductor coupled to the twist conductor; and
causing the twist conductor to change shape by twisting along a length of the twist conductor.
